(19) United States
(12) Patent Application Publication
(10) Pub. No.: US 2010/0298689 A1
(43) Pub. Date: Nov. 25, 2010

(54) SYSTEMS AND METHODS FOR DETECTING FLOW AND ENHANCING SNR PERFORMANCE IN PHOTOACOUSTIC IMAGING APPLICATIONS

(75) Inventor: Yao Wang, Groton, CT (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/742,963

(22) PCT Filed: Nov. 13, 2008
(86) PCT No.: PCT/IB08/54770

§ 371 Date: Jul. 26, 2010

Related U.S. Application Data
(60) Provisional application No. 60/987,841, filed on Nov. 14, 2007.

(51) Int. Cl. A61B 5/05; G06F 19/00; A61B 5/026

(52) U.S. Cl. 600/407; 702/19; 702/48

(57) ABSTRACT

The present disclosure provides systems and methods for combining photoacoustic/thermoacoustic imaging with power Doppler signal processing. More particularly, the disclosed systems and methods involve use of encoded Doppler signals in order to detect and image in vivo blood flow. The disclosed flow detection systems and methods may be used in photoacoustic imaging using PD to achieve, inter alia, enhanced signal-to-noise (SNR) and sensitivity performances. A method for detecting flow in a target region may involve (i) obtaining a encoded signal containing photoacoustic imaging data for the target region using a photoacoustic imaging system, (ii) decoding the encoded signal, (iii) passing the decoded signal through a demodulator and a low-pass filter, resulting in a base-band signal, (iv) passing the base-band signal through a wall filter, resulting in an uncluttered signal; and (iv) estimating the Ro value by integrating the power spectrum of the uncluttered signal.

### CROSS-REFERENCE TO RELATED APPLICATIONS

The present application is related to a pending, commonly assigned provisional patent application entitled “Coded Excitation For Photo-Acoustic and Thermo-Acoustic Imaging” which was filed on Jun. 29, 2007 and assigned Ser. No. 60/947,078. The entire contents of the foregoing provisional patent application are incorporated herein by reference.

### BACKGROUND

1. Technical Field

The present disclosure relates to photoacoustic and thermoacoustic imaging. More particularly, the present disclosure relates to systems and methods for generating spatial distribution of flow in photoacoustic and thermoacoustic imaging applications.

2. Background Art

Blood flow is the fundamental mechanism that carries nutrients, oxygen and regulator proteins to biological tissues and brings back exhausts of metabolism. Angiogenesis is now well-accepted as the single most important factor to sustain cancer growth and proliferation. (See, e.g., J. Folkman, Tumor angiogenesis: therapeutic implications. N Engl J Med. 285:1182-1186, 1971.) In general, this class of hyperaemia condition is associated with a variety of diseases, e.g., arthritis, macular degeneration, endometriosis, and the like. In vivo imaging of such conditions has broad implications. However, such imaging applications are also challenging.

Traditional ultrasound approaches to in vivo imaging of blood flow are complicated and made difficult by the inherently low signal-to-noise ratio (SNR) levels of ultrasound backscatter signals from red blood cells (erythrocytes), which are typically 10-20 dB lower than ultrasound signals from tissue. (See, e.g., K. Shung, Scattering of ultrasound by blood, IEEE Trans. Biomed. Eng.; 23:460-467, 1976). The relatively weak ultrasound backscatter signals are often submerged around or under the noise floor. To assist in the detection of such weak signals, ultrasound developers and manufacturers have developed a vast pool of literature as well as empirical experience. (See, e.g., C. Kasai, K. Namekawa, A. Koyano, and R. Omoto, Real-Time Two-Dimensional Blood Flow Imaging Using an Autocorrelation Technique, IEEE Trans. UFFC, Vol. su-32, No. 3, 1985.)

In ultrasound, while large-vessel blood flow is generally at high velocity and can be readily detected by conventional color Doppler sonography that encodes the mean Doppler frequency shift, blood flow at the micro-vascular level is at a lower velocity and is less readily detectable by this means. Of note, micro-vascular flow is of interest in addressing hyperaemia conditions. As distinguished from conventional color Doppler sonography, power Doppler (PD) sonography encodes the amplitude of the power spectral density of the Doppler signal. (See, e.g., J. M. Rubin, R. O. Bude, P. L. Carson, R. L. Bree and R. S. Adler, Power Doppler US: A Potentially Useful Alternative to Mean Frequency-based Color Doppler US, Radiology, 190:853-856, 1994.) PD sonography is thus a sensitive method for demonstrating the presence of blood flow in small vessels. The PD signal is actually a measure of the density of moving reflectors at a particular level and thus of the fractional vascular volume. (See, e.g., P. C. Taylor, The value of sensitive imaging modalities in rheumatoid arthritis, Arthritis Res Ther.; 5(5): 210-213, 2003.)

Yet, as with other ultrasound techniques, PD ultrasound is insensitive to flow in sub-millimeter vessels and is thus only an indirect surrogate for measurement of capillary flow. This limitation arises primarily from the previously mentioned fact that ultrasound backscatter from blood is much weaker (10-20 dB) than signals from tissue. The resultant weaker signal is primarily due to the concentration of echogenic red blood cells in blood being orders of magnitude lower in density as compared to echogenic cells constituting, for example, liver tissues. (See, e.g., K. Shung, Scattering of ultrasound by blood). There is also frequency dependence of the elastic backscatter (power of 6). (See, e.g., P . Morse, K. Ingard, Theoretical Acoustics, Princeton University Press). However, even at high frequency (e.g., 40 MHz), mouse ventricle still appears as a void in contrast to myocardial muscles (http://www.visualsonicscom). At such high frequency, ultrasound penetration becomes severely limited.

The recent advent of photoacoustic imaging has added a new dimension of opportunity for technological advancement. (See, e.g., X. Wang, Y, Pang, G. Ku, G. Stoica, and L. Wang, Three-dimensional laser-induced photoacoustic tomography of mouse brain with the skin and skull intact, Optical Letters, Vol. 28, No. 19, 1739-1741, 2003.) Generally, photoacoustic imaging uses a short laser pulse to induce rapid heating (due to optical absorption) and follow-up relaxation of a target subject. This mechanical deformation of the subject results in acoustic waves which are then detected by an ultrasound transducer and utilized to form a map of absorber distribution and strength. In contrast with traditional ultrasound approaches, the resultant absorption signal strength for red blood cells targeted during photoacoustic imaging is much higher than the resultant absorption signal strength for tissue. Thus, new opportunities exist for improving microvasculature flow detection using photoacoustic imaging. However, as compared to the well-developed signal processing technology associated with traditional ultrasound approaches, signal processing of weak signals in photoacoustic imaging applications is a relatively undeveloped field. The same can be said for thermoacoustic imaging applications where a laser source is replaced by a microwave antenna.

Current systems and methods utilizing Doppler signal processing (and, more specifically, Doppler frequency shifts) for photoacoustic and thermoacoustic imaging are limited in application and contain many drawbacks. (See, e.g., P. Beard, Blood flow velocity measurement, U.S. Patent Publication No. 2005/0150309 A1, hereinafter referred to as the “Beard Application”). The Beard Application relates generally to photoacoustic flow imaging utilizing the Doppler frequency shift; however, the Beard Application fails to address issues associated with flow detection at lower flow rates. Given photoacoustic penetration capabilities, imaging of extremities and/or imaging at shallow depths, wherein perfusion-type flow is far more prevalent and detection sensitivity to such slow motion is of paramount importance, offer promising applications thereof Thus, the failure of the Beard Application to address imaging of low flow rate regions is clearly disadvantageous. A further limitation of the Beard Application is the prevalence of a flash artifact resulting from tissue motion. The inability of the Beard Application to reduce or eliminate the flash artifact is primarily due to its reliance on Doppler frequency shifts. Other concerns relating to Doppler signal processing include detection angle dependence and the potential aliasing effect at faster flow rates.

Thus, systems and methods are needed that broadly apply Doppler signal processing to photoacoustic imaging applications (including reduced flow rate detection) while avoiding flash artifacts, reducing angle dependence and compensating for the aliasing effect at faster flow rates. A further need exists for photoacoustic imaging systems and methods that are effective in signal processing of relatively weak signals.

### SUMMARY

The present disclosure provides systems and methods that advantageously enhance SNR performance in photoacoustic imaging and thermoacoustic applications. More particularly, the presently disclosed systems and methods involve the generation and use of encoded Doppler signals in order to detect and image in vivo flow, e.g., blood flow. In general, power Doppler (PD) techniques, similar to those used in ultrasound applications, may be used according to the present disclosure to encode the amplitude of the power spectral density of the Doppler signal.

Exemplary embodiments of the present disclosure relate to the use of coded excitation to obtain a Doppler flow signal in order to enhance SNR and sensitivity performances. The disclosed systems and methods have widespread application, including, inter alia, flow detection (particularly at lower flow rates), and disease, disorder or condition diagnosis (e.g., rheumatoid arthritis, age-related macular degeneration, skin cancer/melanoma, esophageal cancer, Barrett's esophagus, vasculitis, benign prostate hyperplasia, prostate cancer, breast cancer, endometriosis, early inflammatory responses associated with atherosclerosis in carotid artery, muscle perfusion in sports medicine, and the like).

By design, the systems and methods disclosed herein combine the benefits of photoacoustic imaging (which advantageously yields stronger optical absorption by blood) with the merits of PD techniques that have been used routinely in clinical diagnostic ultrasound. The resulting systems and methods enable users to perform effective flow detection and/or measurement using photoacoustic imaging, even at low flow rates, e.g. perfusion-type flow.

Additionally, exemplary methods of the present disclosure are effective for detecting flow in a target region of interest, e.g., a blood vessel. Implementations of the disclosed method may advantageously include the steps of (i) obtaining an encoded signal containing photoacoustic imaging data for the target region using a photoacoustic imaging system, (ii) decoding the encoded signal in a manner consistent with the disclosure in a pending, commonly assigned provisional patent application entitled “Coded Excitation For Photo-Acoustic and Thermo-Acoustic Imaging” which was filed on Jun. 29, 2007 and assigned Ser. No. 60/947,078 (previously incorporated herein by reference; the decoded signal generally may take the form of a base-band signal, an analytic signal and/or a raw radio-frequency signal); iii) passing the encoded signal through a demodulator (if it is a radio-frequency signal) and a low-pass filter, resulting in a base-band signal as commonly used in ultrasound imaging and telecommunications systems, (iv) passing the base-band signal through a wall filter, thereby removing all stationary signals that remain virtually constant over time, and (v) estimating the R₀ value by integrating the power spectrum of the signal resulting from the wall filter.

The disclosed wall filter is generally effective to remove tissue signal (sometimes referred to as clutter elsewhere in ultrasound and radar imaging literature) from the decoded signal in base-band. In exemplary embodiments of the present disclosure, the R₀ value advantageously functions as an estimate of bulk blood flow. Indeed, the R₀ value may be used to detect hyperemia conditions. Of note, the R₀ value may be estimated using the following calculation:

R₀ =∫z(t)z*(t)dt=∫(x²(t)+y²(t))dt=∫P(ω)dω,

wherein z is the uncluttered signal and z(t)=x(t)+iy(t) and P is the power spectrum of the uncluttered signal.

The present disclosure further provides advantageous photoacoustic and thermoacoustic imaging systems. In an exemplary embodiment thereof, the photoacoustic/thermoacoustic imaging system includes: (i) one or more electromagnetic beam sources, e.g., laser and/or microwave, adapted to irradiate a target location, wherein the power spectrum of the one or more beams is encoded; (ii) one or more ultrasound detectors adapted to detect a photoacoustic signal that includes a base-band signal resulting from a target sample; (iii) means for synchronization of irradiation and detection functionalities, and (iv) means for processing the photoacoustic signal to derive flow information related to the target location.

The means for processing the photoacoustic/thermoacoustic signal may include a demodulator, a low-pass filter, a wall filter and means for R₀ estimation. The demodulator and low-pass filter are generally adapted to demodulate and extract the base-band signal of the photoacoustic signal. The wall filter is typically adapted to remove tissue clutter from the base-band signal of the photoacoustic signal. The means for R₀ estimation is generally associated with a processing unit/computer, and is adapted to estimate bulk flow by integrating the power spectrum of the uncluttered base-band signal.

The one or more electromagnetic beam sources generally take the form of laser(s), e.g., a semi-conductor based laser source, and/or microwave(s), e.g. radio-frequency microwave antennas. Typically, the electromagnetic beam sources operate at a low pulse-repetition frequency. The ultrasound detectors include one or more transducer arrays. The means for synchronization and the means for processing are typically reposited on and operated by a processing unit/computer. Indeed, the means for synchronization and means for processing may be hardware- and/or software-based.

In exemplary implementations of the present disclosure, the target sample is a blood vessel and the R₀ value is used to detect hyperemia conditions. However, the disclosed systems/methods have broad-based applications and offers many advantages over prior art as discussed in the present disclosure. Additional features, functions and benefits of the disclosed systems and methods will be apparent from the description which follows, particularly when read in conjunction with the appended figures.

### BRIEF DESCRIPTION OF THE DRAWINGS

To assist those of ordinary skill in the art in making and using the disclosed systems and methods, reference is made to the appended figures, wherein:

FIG. 1 schematically depicts an exemplary technique for performing power Doppler with photo-acoustics according to the present disclosure; and

FIG. 2 depicts an exemplary power Doppler processing sequence.

### DESCRIPTION OF EXEMPLARY EMBODIMENT(S)

As noted herein above, the present disclosure provides systems and methods that advantageously combine photoacoustic and thermoacoustic imaging with power Doppler (PD) technology, e.g., to detect reduced flow rates with enhanced signal-to-noise (SNR) performance.

Of note, coded excitation has been employed to increase SNR of transmitted signals, e.g., in the telecommunications field. In particular, binary sequences may be used to encode signals, which are transmitted through a medium subject to noise or interference. The signals are received and decoded to recover medium information. Systems and methods which combine the use of coded excitation with the benefits of the well known Doppler effect (particularly in photoacoustic imaging applications) could prove highly beneficial for weak signal processing. In this regard, reference is made to a commonly assigned, co-pending provisional patent application entitled Coded Excitation For Photo-Acoustic and Thermo-Acoustic Imaging (Ser. No. 60/947,078, filed Jun. 29, 2007, the contents of which were previously incorporated herein by reference).

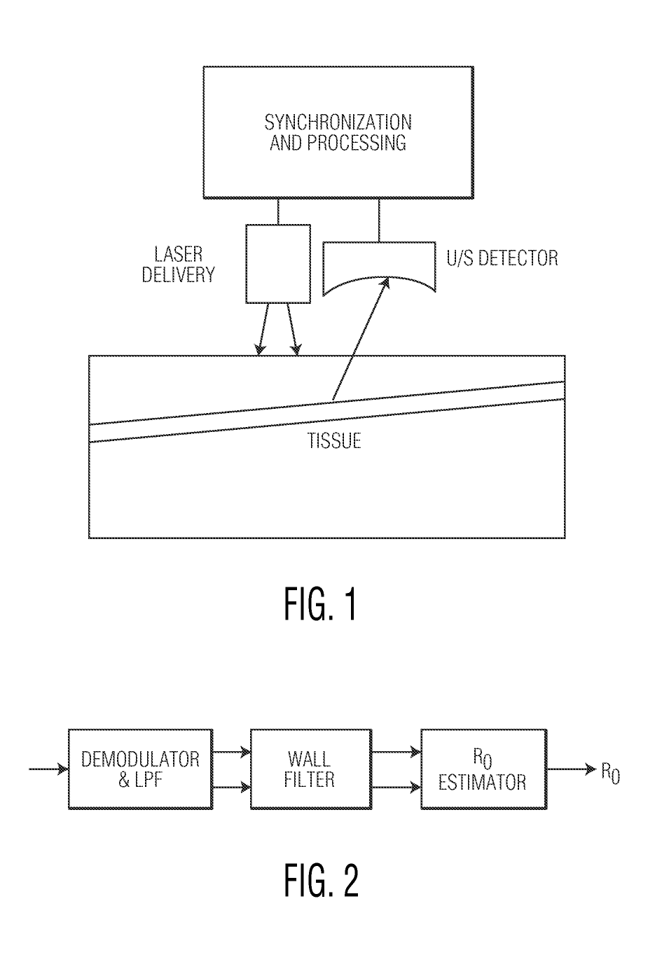

With initial reference to FIG. 1, an exemplary set-up of a photo-acoustics system for performing in vivo tissue imaging is schematically depicted. Laser irradiation is synchronized with ultrasound receiving components such that time origin for recorded photo-acoustic signals is aligned with the instant of laser delivery. Once light is delivered, chromophores (e.g., red blood cells in flowing blood) absorb the energy. Upon absorption of such light energy (e.g., laser irradiation), the chromophores rapidly expand and then relax. This phenomenon creates a disturbance in the medium and results in and/or generates a photo-acoustic wave that may be detected by an ultrasound detector.

According to the presently disclosed systems and methods, recorded photoacoustic/thermoacoustic signals are beamformed to form spatial maps of the absorbers and then processed according to the functional diagram in FIG. 2. Indeed, as shown in FIG. 2, beamformed signals from consecutive frames are first demodulated and sent through a low-pass filtered to extract the base-band signals. According to the present disclosure, either an analytic signal (one-side Hilbert transform of the recorded photo-acoustic signal) or raw radio-frequency (RF) signal may be used. After demodulation and low-pass filtration, a wall filter is typically used to remove tissue clutter from this processed signal. Finally, R₀ estimation produces a power estimate which may be translated to or correlated with bulk flow properties/parameters.

In the presently disclosed systems and methods, the sensitivity to reduced flow generally comes from the noted R₀ estimate. In diagnostic ultrasound, this calculation is based on the radio-frequency signal, the base-band signal or the analytical signal. Thus, the zero-lag auto-correlation of the signals is computed from consecutive observations. According to the present disclosure, R₀ is computed by integrating the power spectrum as shown in Equation 1:

R₀ =∫z(t)z*(t)dt=∫(x²(t)+y²(t))dt=∫P(ω)dω (1)

wherein z is the signal and z(t)=x(t)+iy(t) and P is the power spectrum of the signal. (See, e.g., C. Kasai, K. Namekawa, A. Koyano, and R. Omoto, Real-Time Two-Dimensional Blood Flow Imaging Using an Autocorrelation Technique, IEEE Trans. UFFC, Vol. su-32, No.3, 1985.)

Thus, since the entire spectrum of the signal is taken into account (rather than just a small frequency shift), the calculated R₀ has demonstrated superior sensitivity to reduced flow rates. An associated benefit, also observed in ultrasound applications of PD, is an increased display dynamic range wherein gain control can be lowered to the level of electronic noise. (See, e.g., P. C. Taylor, The value of sensitive imaging modalities in rheumatoid arthritis, Arthritis Res Ther.; 5(5): 210-213, 2003.)

By adapting PD techniques to photoacoustic imaging, the present disclosure is distinct from more conventional Doppler ultrasound measurement. Indeed, the present disclosure generally involves detection of the presence of bulk flow rather than blood velocity. The presently disclosed systems and methods thus offer many advantages relative to prior art/conventional techniques and systems, some of which are herein discussed:

(i) With the currently disclosed systems and methods, aliasing effect is substantially reduced or eliminated. Disadvantageous aliasing effects are encountered, for example, in pulsed-wave Doppler imaging where pulse repetition frequency (PRF) is not sufficiently high to accommodate faster flow rates.

(ii) Since the disclosed systems and methods measure bulk flow, and not velocity, there is virtually no angle dependence and, accordingly, no reliance on the Doppler angle to induce a frequency shift. By contrast with frequency shift approaches, the presently disclosed systems and methods measure the number and strength of visible scatterers, none of which depends on velocity and shear rate. A slight angle dependence may still exist according to the present disclosure because flow signal with a frequency shift close to zero, e.g. an observation angle orthogonal to flow, may be eliminated or attenuated by, for example, a wall filter. Despite this potential impact of a wall filter, in practical implementations it is generally desirable to include a wall filter, e.g., to reject or dampen soft tissue motion clutter. The overall effect of a wall filter on angle dependence, however, may be expected to be very small due to the spectral broadening resulting from the aperture/scan-head, the flow profile, and the signal bandwidth.

(iii) The systems and methods herein disclosed are able to use the lowest possible pulse repetition frequency (PRF), thereby reducing the potential for flash artifacts from tissue motion compared to techniques that rely on Doppler frequency shift.

(iv) The presently disclosed systems and methods also result in favorable SNR performance based, at least in part, on the fact that optical absorption of photoacoustic signal by blood is typically orders of magnitude greater than the corresponding optical absorption for tissue.

(v) Since power Doppler (PD) techniques are not based on frequency shifts, the presently disclosed systems and methods are sensitive to reduced flow rates as compared to prior art/conventional systems. This sensitivity enables imaging of, e.g. extremities, shallow depths, and other applications/locations where perfusion-type flow is prevalent.

(vi) The disclosed systems and methods also facilitate elimination of the skin line present in photoacoustic and thermoacostic imaging. In conventional photoacoustic imaging, the skin line is always present due to the non-homogeneity between a coupling medium (e.g., gel or water) and tissue. The skin line hinders visualization of targets-of-interest adjacent or close to the surface of the skin. Since photoacoustic imaging using PD only visualizes moving targets, skin line elimination may be advantageously achieved.

There exist many potential applications for the systems and methods disclosed herein. One particularly advantageous application relates to visualization and quantification of perfusion type flow typically associated with hyperemia conditions. Considering the penetration capabilities of photoacoustic imaging, target regions for visualization and quantification are generally relatively shallow (wherein depth is measured from the detection instruments, whether positioned externally or internally, e.g., based on endoscopic/laparoscopic techniques). Diseases, disorders and conditions that may be detected, monitored and/or measured according to the present disclosure include, but are not limited to: rheumatoid arthritis, age-related macular degeneration, skin cancer/melanoma, esophageal cancer, Barrett's esophagus, vasculitis, benign prostate hyperplasia, prostate cancer, breast cancer, endometriosis, early inflammatory responses associated with atherosclerosis in carotid artery, muscle perfusion in sports medicine, and the like.

According to exemplary implementations of the present disclosure, the penetration depth for photoacoustic imaging is generally on the order of a few centimeters, depending on factors such as tissue conditions and the like. At such depths, perfusion type flow is typically more pertinent. Additionally, the low pulse-repetition frequency (PRF) associated with solid-state laser technology/implementations makes reduced flow imaging more practical. Semi-conductor based laser sources, e.g. laser diodes, may enable high PRF operations for photo-acoustics, which may be used to improve the image quality as described herein. Moreover, this disclosure is also applicable to thermo-acoustic imaging where microwave sources are used.

Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof

1. A method for detecting flow in a target region, the method including the steps of:
a. obtaining an encoded signal containing photoacoustic imaging data for the target region using a photo acoustic imaging system;
b. decoding the encoded signal;
c. passing the decoded signal through a demodulator and a low-pass filter, resulting in a base-band signal;
d. passing the base-band signal through a wall filter, resulting in an uncluttered base-band signal; and
e. estimating an R₀ value for an entire spectrum of the uncluttered base-band signal by integrating a power spectrum of the uncluttered base-band signal wherein the R₀ value provides a power estimate that can be translated to or (ii) correlated with bulk flow properties/parameters in the target region not based on Doppler frequency shifts.

2. The method according to claim 1, wherein the decoded signal is a base-band signal.

3. The method according to claim 1, wherein the decoded signal is an analytic signal.

4. The method according to claim 1, wherein the decoded signal is a raw radio-frequency signal.

5. The method according to claim 1, wherein the wall filter removes tissue clutter from the decoded signal.

6. The method according to claim 1, wherein the R₀ value is an estimate of bulk blood flow.

7. The method according to claim 1, wherein the R₀ value is used to detect hyperemia conditions.

8. The method according to claim 1, wherein the R₀ value is estimated using the following calculation:
R₀ =∫z(t)z*(t)dt=∫(x²(t)+y²(t))dt=∫P(ω)dω,
wherein z is the uncluttered base-band signal and z(t)=x(t)+iy(t) and P is the power spectrum of the uncluttered base-band signal.

9. A photoacoustic imaging system comprising:
a. one or more electromagnetic beam sources adapted to irradiate a target location, wherein the power spectrum of the one or more beams is encoded;
b. one or more ultrasound detectors adapted to detect one of a photoacoustic signal or a signal that includes a an encoded base-band signal resulting from a target sample;
c. means for synchronization of irradiation and detection functionalities, and
d. means for processing the encoded base-band signal to derive flow information related to the target location, wherein processing the encoded base-band signal includes estimating an R₀ value for an entire spectrum of a corresponding decoded base-band signal wherein the R₀ value provides a power estimate that can be (i) translated to or (ii) correlated with bulk flow properties/parameters in the target region not based on Doppler frequency shifts.

10. The system according to claim 9, wherein the means for processing the encoded base-band signal includes a demodulator, a low-pass filter, a wall filter and means for R₀ value estimation.

11. The system according to claim 10, wherein the demodulator and low-pass filter are adapted to decode and extract a base-band signal of a corresponding photoacoustic/thermoacoustic signal.

12. The system according to claim 10, wherein the wall filter is adapted to remove tissue clutter from a base-band signal of a corresponding photoacoustic/thermoacoustic signal.

13. The system according to claim 10, wherein the means for R₀ estimation is adapted to estimate bulk flow by integrating a power spectrum of an uncluttered base-band signal.

14. The system according to claim 9, wherein the decoded base-band signal is one of: (i) an analytic signal, and (ii) a raw radio-frequency signal.

15. The system according to claim 9, wherein the one or more electromagnetic beam sources includes a laser and/or a microwave.

16. The system according to claim 9, wherein the one or more electromagnetic beam sources operate at a low pulse-repetition frequency.

17. The system according to claim 9, wherein the one or more electromagnetic beam sources is a semi-conductor based laser source and/or a microwave antenna.

18. (canceled)

19. The system according to claim 9, wherein the means for synchronization and the means for processing are reposited on and operated by a processing unit/computer.

20. The system according to claim 9, wherein the means for synchronization and means for processing are hardware-based.

21. (canceled)

22. The system according to claim 9, wherein the R₀ value is used to detect hyperemia conditions.
